(19) United States
(12) Patent Application Publication
(10) Pub. No.: US 2021/0122781 A1
(43) Pub. Date: Apr. 29, 2021

(54) STAPLED PEPTIDES

(71) Applicant: Agency for Science, Technology and Research, Singapore (SG)

(72) Inventors: Tsz Ying Yuen, Singapore (SG); Charles William Johannes, Singapore (SG); Fernando Jose Ferrer Gago, Singapore (SG)

(21) Appl. No.: 16/497,301

(22) PCT Filed: Mar. 26, 2018
(86) PCT No.: PCT/SG2018/050135

§ 371 Date: Sep. 24, 2019

(30) Foreign Application Priority Data
Mar. 24, 2017 (SG) 10201702441Q

(51) Int. Cl. C07K 1/113

(52) U.S. Cl. CPC C07K 1/113

(57) ABSTRACT

This invention relates to a process for producing a compound of Formula I, comprising: 1) performing a stereoselective metathesis reaction on a compound of Formula II so as to form an intramolecular alkenyl chain, and 2) cleaving S from P² so as to produce a compound of Formula I. A product containing an (Z)- or (E)-olefin isomer stabilised in an a-helical conformation is obtained by the said process.

### FIELD

This invention relates to the stereoselective formation of stapled peptides.

### BACKGROUND

The concept of helix stabilisation by cross metathesis of 2 amino acid sidechains was first introduced by Grubbs et al (Agnew. Chem., Int. Ed. 1998, 37, 3281). This technology, later termed “stapled peptides”, was further refined with the introduction of an all-hydrocarbon bridge. Whilst it is well known that i, i+4 stapled peptides are formed exclusively as the cis isomer, the issue of cis/trans selectivity is rarely examined for i, i+7 stapled peptides. It has been previously found that these i, i+7 stapled peptides consistently give rise to cis/trans olefin mixtures in which the less polar adduct was more biologically active.

Traditionally, stapled peptides are synthesised from their corresponding linear peptides by solid-phase ring closing metathesis using Grubbs I catalyst in dichloroethane (DCE). More recently, a procedure involving the use of Hovey-Grubbs II catalyst was also developed (FIG. 1).

Stereoselective ring-closing olefin metathesis has been an active area of research for some time in the fields of organometallics and organic synthesis. Since the discovery that some ruthenium catalysts showed marginal product selectivity, tremendous efforts have been dedicated to establishing factors that govern selectivity. As a result, a novel class of chelated ruthenium catalysts was developed. Not only have they found applications in organic synthesis, but the use of the “Grubbs Z-selective catalyst” has also been shown to provide an i, i+7 stapled peptide in high yield and selectivity (85% conversion, 90% Z).

Although early metathesis catalysts comprised the more reactive molybdenum metal, Mo-systems are largely disfavoured due to their high air/moisture sensitivity and the need to set up reactions in a glovebox. By formulating the active Mo complexes in microcrystalline wax, these reagents (CatPac-1, CatPac-3, FIG. 2) were rendered air stable in pellet form. These catalysts enable Z-selective cross-metathesis reactions, with CatPac-3 being more selective than CatPac-1.

Investigations towards more potent i, i+7 p53-targeting stapled peptides have found that the less polar stapled peptide isomer consistently performs better in biological assays. However, under standard stapling conditions with Grubbs I catalyst in DCE, cis and trans isomers are formed in approximately equal amounts, meaning that any yield is reduced by half, as the more polar stapled peptide is not desirable due to the lower biological response. The current state-of-the-art is provided in Table 1.

TABLE 1
Current catalyst products and disadvantages in producing stapled peptides

| Catalyst | Product Composition | Disadvantages |
|---|---|---|
| Grubbs 1 | cis + trans isomers (1:1) | discard half the material |
| Hoveyda-Grubbs II | single isomer? (to be investigated) | unknown stereoselectivity |
| Grubbs Z | cis isomer only | resin incompatibility |
| CatPac Mo-catalysts | cis isomer only | air/moisture sensitivity in |
| | | solution, specialized |
| | | glassware required |

There is currently a lack of accurate reporting of cis/trans ratios and stereoselective strategies that allow access to a single stapled peptide configurational isomer. Consequently, conventional strategies resulting in a mixture of isomers also requires the discarding of half the newly synthesised material when the need for only one isomer arises, such as when there is a difference in biological profiles. Other known methods and catalysts that favour a single isomer currently favour the less biologically active cis-isomer.

An important step in stapled peptide synthesis is the ruthenium-catalysed olefin metathesis. Although this process allows the formation of some of the largest macrocycles with excellent efficiency, E/Z selectivity is often unpredictable. Hence, there is a need to maximise the product yield of the more biologically active stapled peptide isomer without compromising product purity, cost or ease of operation.

### SUMMARY OF INVENTION

In a first aspect of the present invention there is provided a process for producing a compound of Formula I

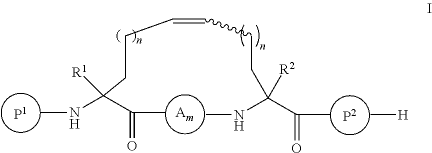

said process comprising the steps of:

a) performing a stereoselective metathesis reaction on a compound of Formula II

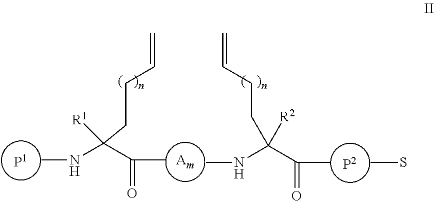

so as to form an intramolecular alkenyl chain, and;

b) cleaving S from P² so as to produce a compound of Formula I;

wherein:

m is an integer between 1 and 8;

each n is independently an integer between 0 and 12;

each A is independently an amino acid residue;

R¹ and R² are each independently alkyl;

P¹ and P² are each independently either an amino acid residue or an oligopeptide chain or a polypeptide chain, wherein P¹ has a terminal amino group and P² has a terminal carboxyl group;

[symbol] is a carbon-carbon single bond that is attached to a carbon atom of the double bond such that the compound of Formula I is in either the (E)-isomer configuration or the (Z)-isomer configuration or is a mixture of these; and,

S is a solid state resin.

The following options may be used in conjunction with the first aspect, either individually or in any suitable combination.

The process may convert at least 90% of the compound of Formula II into a compound of Formula I, or at least 91, 92, 93, 94, 95, 96, 97, 98 99, 99.5 or 99.9%. The process may produce a compound of Formula I with a geometric stereoisomer purity of greater than 50%, or greater than about 55, 60, 65, 70, 75, 80, 85, 90, 95, 96, 97, 98, 99, 99.5, or 99.9%. It may produce a product in which more than about 50%, or more than about 55, 60, 65, 70, 75, 80, 85, 90, 95, 96, 97, 98, 99, 99.5, or 99.9% of the product formed in step a) is the less-polar stereoisomer. The less-polar stereoisomer may be the (E)-stereoisomer. For example, the process may result in a 95% conversion and 65% geometric stereoisomer purity, or it may result in 60 to 80% conversion and 60 to 80% geometric stereoisomer purity, or 80 to 95% conversion and 65-85% geometric stereoisomer purity, or 70 to 99% conversion and 60 to 95% geometric stereoisomer purity.

In the compound of Formula II, m may be an integer between 1 and 6, or between 3, and 6, 2 and 5, 3 and 4, 1 and 5 or 2 and 4, e.g., it may be 1, 2, 3, 4, 5 or 6. When m is 2, the chiral centre attached to R¹ may be R and the chiral centre attached to R² is S, or the chiral centre attached to R¹ may be S and the chiral centre attached to R² is R. When m is 3, the chiral centres attached to both the R¹ and R² are the same, either both R or both S. Each A may independently be a naturally occurring L-α-amino acid, or at least one A may be an unnatural amino acid. R¹ and R² may both be methyl. Both P¹ and P² may comprise at least one naturally occurring L-α-amino acid. Both P¹ and P² may comprise at least one unnaturally occurring amino acid.

The compound of Formula II comprises a peptide chain bound to a solid state resin. The compound of Formula II may be produced via solid-state peptide synthesis. The intramolecular alkyl linker is formed before cleavage of the peptide from solid state resin. The solid state resin, S in Formula II, may be a polymeric material, for example it may be polystyrene, or it may be polyamide, or it may be polyethylene glycol, or it may be a blend of two or more of these polymers.

The process is conducted in two steps, (a) and (b). The metathesis step of step (a) may be conducted in the presence of a catalyst and an organic solvent. The catalyst may be a non-anchored catalyst. It may be a non-anchored alkylidene catalyst. It may comprise ruthenium. It may for example be any one of a Grubbs I catalyst, a Grubbs II catalyst, a Hoveyda-Grubbs I catalyst, a Hoveyda-Grubbs II catalyst, a Grubbs Z catalyst or a mixture of any two or more of these. The metathesis reaction, step (a), may use a single aliquot addition of catalyst, or it may use multiple aliquots of fresh catalyst added to the reaction mixture. It may use 2, 3, 4, 5 or more than 5 aliquot additions. The solvent present for the metathesis reaction may be a halogenated alkane. It may be dichloroethane. The metathesis reaction may be conducted at a temperature between about 15° C. and about 30° C. It may be conducted at a room temperature. It may be conducted at a temperature between about 40° C. and about 60° C. The process of step (a) may convert at least about 55% of the compound of Formula II into the compound of Formula I.

The compound of Formula I and compound of Formula II may both have P¹, P² and A groups that independently comprise a single amino acid, an oligopeptide chain, or a peptide chain. Each amino acid in each of the P¹, P² and A groups in each compound may be a natural L-α-amino acid or an unnatural amino acid or a derivative thereof. The number of amino acid residues in the A group of both compounds is defined by m. The compound of Formula I and compound of Formula II may both have m between 1 and 6. They may both have a chiral centre on that carbon atom to which the R¹ group is bound, and a chiral centre on the carbon atom to which the R² group is bound. Each chiral centre may independently be either (R) or (S). Compounds of both Formula I and Formula II have an alkyl R¹ group and an alkyl R² group. Each R¹ and R² group may for example be independently methyl, ethyl, propyl or pentyl. Each R¹ and R² group may be a branched alkyl group, for example isopropyl, sec-butyl, tert-butyl or sec-pentyl. Each R¹ and R² group may be a cycloalkyl group, for example cyclopropyl, cyclobutyl, cyclopentyl or cyclohexyl. Both compounds may have a combined number of amino acid residues (i.e., the sum of the number of residues of P¹+P²+m+2) that is between 5 and 20, or between 5 and 15, 10 and 20, 5 and 10 or 10 and 15, e.g., it may be 5, 6, 7, 8, 9, 10, 11, 12, 13, 14, 15, 16, 17, 18, 19, 20.

In one embodiment, the process of the present invention involves producing a compound of Formula I

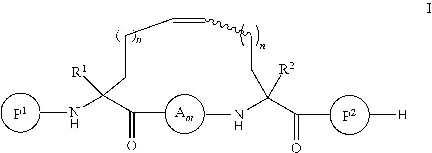

said process comprising performing a stereoselective metathesis reaction on a compound of Formula II

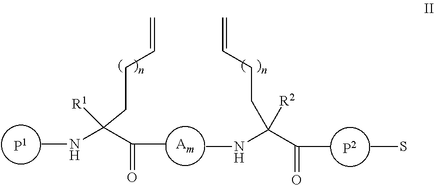

so as to form an intramolecular alkenyl chain, and then cleaving the solid state resin from P² so as to produce a compound of Formula I. The metathesis reaction is catalysed by a non-anchored Grubbs II catalyst dissolved in a halogenated alkane. In both Formula I and Formula II, m is 5, R¹ and R² are both methyl, the n closest to P¹ is 2 and the other n is 5, the chiral centre of the carbon atom attached to R¹ is (S) and the chiral centre of the carbon atom attached to the R² group is (R) and S is a polyethylene glycol resin.

In another embodiment, the process of the present invention involves producing a compound of Formula I

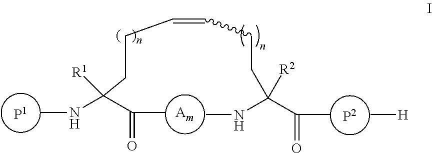

said process comprising performing a stereoselective metathesis reaction on a compound of Formula II

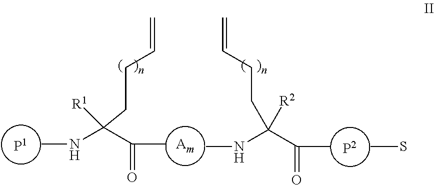

so as to form an intramolecular alkenyl chain, and then cleaving the solid state resin from P² so as to produce a compound of Formula I. The metathesis reaction is catalysed by a non-anchored Grubbs II catalyst dissolved in a halogenated alkane. In both Formula I and Formula II, m is 6, R¹ and R² are each methyl, the n closest to P¹ is 2 and the other n is 5, the chiral centre of the carbon atom attached to R¹ is (S) and the chiral centre of the carbon atom attached to the R² group is (R) and S is a polyethylene glycol resin.

In a second aspect of the present invention, there is provided a product obtained by the process of the first aspect. This product may comprise a compound of Formula I, wherein at least about 50% of the product contains a carbon-carbon double bond in the (E)-stereoisomer configuration. The product may be a peptide analogue stabilised in an α-helical conformation.

### BRIEF DESCRIPTION OF DRAWINGS

FIG. 1. Representative examples of chelated ruthenium catalysts, including the Grubbs Z-selective catalyst.

FIG. 2. Structures of CatPac-1 and CatPac-3.

FIG. 3. Diagrammatic representation of a stapled peptide forming an intramolecular alkenyl linker.

FIG. 4. Metathesis progress and selectivity as determined by reverse-phase HPLC, whereby the late-eluting isomer is the most biologically-active isomer.

FIG. 5. Spleen samples of wildtype-p53 mice harvested 5 hours after intraperitoneal injection with either DMSO (vehicle) or the early or late eluting isomer of stapled peptides VIP82, VIP115 or VIP116. The samples were stained to show p53 expression, which was higher in the late eluting isomer of VIP82 and VIP115 compared to the early eluting isomer, and the same for both isomers for the VIP116 peptide. The vehicle did not show any effect on p53 expression.

FIG. 6. Photomicrographs of (A) spleen and (B) tumour samples from C57BL/6 mice after treatment with DMSO vehicle or p53-stabilising stapled peptide isomers VIP116 (both early-eluting and late-eluting isomers) and ATSP-7041 (late-eluting isomer only).

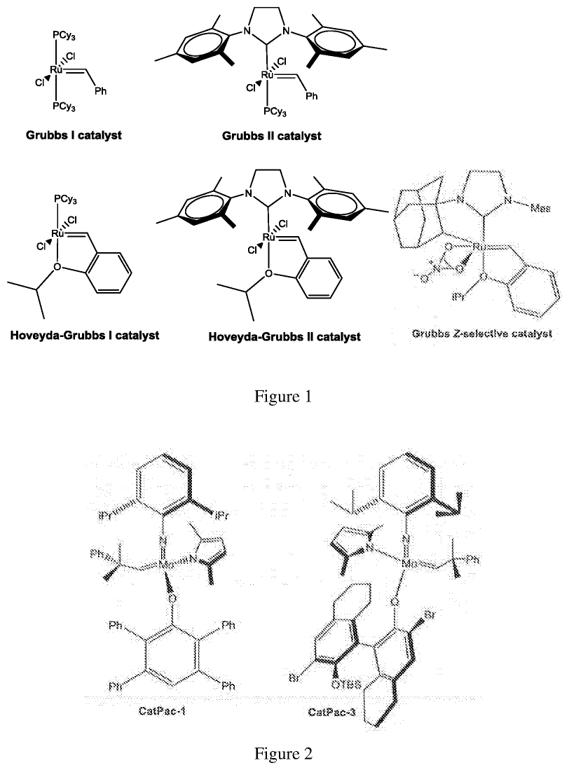

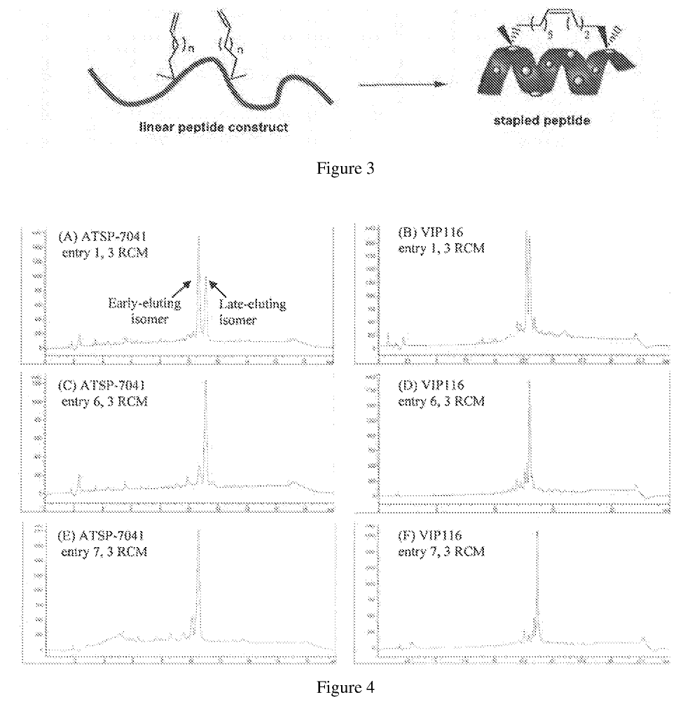

### DEFINITIONS

The term “modified amino acid” as used herein refers to an amino acid that has been chemically modified so as to have an alkenyl side chain, where the alkenyl side chain is an alkyl chain that terminates in an alkene moiety.

The terms “stapled peptides” and “peptide analogues” as used herein refer to peptidic or peptide-like chains that incorporate two or more modified amino acids, such that when the peptide chain becomes a stapled peptide, the alkyl chains of the modified amino acids are covalently joined to produce an intramolecular alkenyl linker that constricts at least a portion of the peptidic chain in at least one conformation.

The terminology “i,i+4” and “i, i+7” as used herein refer to the relative positions of the modified amino acid residues in the peptide chain in relation to each other, in which a first modified amino acid residue is at position i, and the second modified amino acid residue is located a defined number of residues away in the chain. For example, an i,i+4 stapled peptide contains 3 amino acid residues between one modified amino acid residue (e.g., i) and the other modified amino acid residue (e.g., i+4) which is the fourth residue away from the i residue.

The term “stereoselectivity” as used herein refers to the tendency of a chemical reaction to produce one stereoisomer preferentially.

The term “oligopeptide”, as used herein, refers to a peptide chain of between about 2 and about 20 amino acid residues. The related term “polypeptide” as used herein refers to peptide chains that are greater than 20 amino acid residues in length, commonly up to about 50 residues in length.

The term “metathesis reaction”, as used herein, refers to a reaction in which two alkenes are converted to two new alkenes by the exchange of carbon-carbon double bonds, commonly in the presence of an alkylidene catalyst.

The term “anchored”, as used herein in reference to the alkylidene catalyst which forms the alkenyl linker, refers to a catalyst that comprises at least one bidentate or polydentate ligand coordinated to the catalytic metal centre, for example, the Hoveyda-Grubbs, Hoveyda-Grubbs II and Grubbs Z-selective catalysts of FIG. 1 are “anchored” catalysts. Likewise, the term “non-anchored” catalyst, as used herein, refers to a catalyst that does not comprise a bidentate or polydentate ligand, but rather contains all monodentate ligands coordinated to the catalytic metal centre. The terms “ligand”, “bidentate” and “monodentate” all have the usual meanings that are well-known in coordination chemistry.

The term “alkenyl”, as used herein, refers to a hydrocarbon radical derived from an alkene.

The term “between”, as used herein, in reference to a range of values, includes the stated end points. Thus, “between” 1 and 6 includes 1, 2, 3, 4, 5 and 6.

The term “comprises” means “includes”. Variations on the word “comprises”, such as “comprising” and “comprise”, have corresponding meanings. As used herein, the terms “including” and “comprising” are non-exclusive. As used herein, the terms “including” and “comprising” do not imply that the specified integer(s) represent a major part of the whole.

The term “consists essentially of” means “to the exclusion of other additional components purposefully added”, or “only the following recited elements are intended to be present”. Additional components may be present in the defined composition or device provided that they are not intentionally present.

### DESCRIPTION OF EMBODIMENTS

The invention disclosed herein describes the use of Grubbs II catalyst to selectively access the less polar stapled peptide isomer. The conditions outlined herein are highly selective for the less polar isomer of p53-targeting stapled peptides, whilst being tolerant of a diverse range of amino acids with various bulk and charges. In addition to enhanced stereoselectivity, the overall reactivity of the macrocyclisation step is also improved.

The present invention relates to a process for stereoselectively producing a stapled peptide. The stapled peptide produced may be more biologically active than the opposite stereoisomer that is not produced. The stapled peptide isomer may be the trans isomer. As will be described in greater detail below and with reference to the Examples, the methods and catalysts may be used to selectively and efficiently produce stapled peptides in a biologically-active configuration.

### Peptide Chain

The stapled peptides referred to herein are peptides which comprise an intramolecular alkenyl linker between two different residues on the same peptidic chain. The linker constrains the peptide to a particular confirmation, with the strength of the constraint depending on a number of factors, including the size of the peptidic chain and the number of amino acid residues between the ends of the linker.

In order to produce a stapled peptide chain, a peptide chain is first formed that comprises at least two modified amino acid residues capable of being covalently linked. The modified amino acids can be incorporated into a peptide chain by using standard peptide synthesis methods such as solid-phase peptide synthesis which are well-known in the art.

Once the peptide chain is formed, it will contain at least two modified amino acid residues that are a defined distance apart. For instance, the modified amino acid residues may be separated by 6 amino acid residues to form an i, i+7 stapled peptide, or they may be separated by 4 amino acid residues to form an i, i+5 stapled peptide. Other appropriate arrangements of residues will be known by the skilled addressee, or may become known.

In the present invention, the peptide chain to which an intramolecular linker is generated to form a stapled peptide is represented by Formula II:

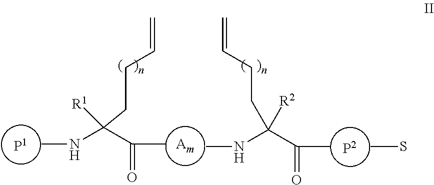

In the compound of Formula II, m is an integer between 1 and 8, or between 1 and 6, 2 and 7, 3 and 4, 2 and 5 or 5 and 8, e.g., 1, 2, 3, 4, 5, 6, 7, or 8. Each n is independently an integer between 0 and 12, or between 0 and 6, 6 and 12, 4 and 8, 2 and 10 or 4 and 12, e.g., 0, 1, 2, 3, 4, 5, 6, 7, 8, 9, 10, 11 or 12. Each A is independently an amino acid residue, which may be either a naturally occurring amino acid or an unnaturally occurring amino acid. R¹ and R² are each independently alkyl, and P¹ and P² are each independently either an amino acid residue (e.g., a natural amino acid or an unnatural amino acid) or an oligopeptide chain or a polypeptide chain, wherein P¹ has a terminal amino group and P² has a terminal carboxyl group. S is a solid state resin from which the final peptide will be cleaved.

Once a peptide chain of Formula II is formed, two further reaction steps are performed so as to produce a stapled peptide.

Firstly, in step (a), the two alkenyl chains are coupled to form the linker. One approach to joining these two alkyl chains together, and hence constraining the peptide, is to use at least one ruthenium- or molybdenum-based catalyst to catalyse a metathesis reaction. Such reactions are well known in the art of hydrocarbon chemistry. The resultant intramolecular alkenyl chain contains a single carbon-carbon double bond at the site where the two chains were joined together, with the remainder of the linker chain being saturated alkyl carbons. The carbon-carbon double bond may be in either the (E)-configuration or the (Z)-configuration. As discussed in more detail below, it is this reaction to join the two alkyl chains together to form a stapled peptide from a peptide chain, and to direct the stereochemistry of the resultant stapled peptides, that is the focus of this disclosure.

Secondly, in step (b), the formed stapled peptide is cleaved from the solid state resin using commonly known reagents such as hydrogen fluoride or trifluoroacetic acid, to produce a free compound of Formula I with a protonated C-terminal. The solid state resin may be a polymeric material. It may for example be polystyrene, polyamide, polyethylene glycol, a polyethylene glycol resin, or it may be a blend of two or more than two of the above polymers.

### Peptide Stapling Method

As discussed above, a stapled peptide is formed in step a) from a peptide chain of Formula II when the alkenyl chains are reacted and joined together via a catalysed metathesis reaction. Following cleavage from the solid state resin (i.e., step (a) described above) a compound of Formula I as defined earlier herein is formed.

The peptide backbone of the stapled peptide has three regions comprising non-modified amino acids, or at least amino acids that are not involved in forming the intramolecular linker. These three regions are defined in both Formula I and Formula II as P¹, P² and A. P¹ may be an amino acid, or it may be an oligopeptide sequence, or it may be a polypeptide sequence. The P¹ residue or chain comprises one residue that is the N-terminus for the stapled peptide. The P¹ chain terminates in either a free amine group or a protected amine group. P² may also be an amino acid, or it may be an oligopeptide sequence, or it may be a peptide sequence. The P² residue or chain comprises one residue that is the C-terminus for the stapled peptide. The P² chain terminates in a carboxylic acid or an amide group. When P¹ and P² are peptide chains, there is no limit as to the length of either of these chains, provided that there is at least one residue present in each. A may be an amino acid, or it may be an oligopeptide sequence, or it may be a polypeptide sequence. A may comprise between 1 and 8 amino acid residues, as defined by m. Hence, m may be an integer between 1 and 8, for example between 1 and 6, 1 and 4, 4 and 8, 3 and 7 or 2 and 6, e.g., 1, 2, 3, 4, 5, 6, 7 or 8. The number of residues in A is limited by the maximum length of the linker able to be formed, and the linker must traverse the distance of A in order to form the stapled peptide.

The amino acids of the P¹, P² and A groups, whether a single residue, an oligopeptide or a polypeptide, may each be selected from a naturally occurring L-α-amino acid (e.g., L-α-arginine, L-α-histidine, L-α-lysine, L-α-aspartic acid, L-α-glutamic acid, L-α-serine, L-α-threonine, L-α-asparagine, L-α-glutamine, L-α-cysteine, L-α-selenocysteine, L-α-glycine, L-α-proline, L-α-alanine, L-α-valine, L-α-isoleucine, L-α-leucine, L-α-methionine, L-α-phenylalanine, L-α-tyrosine or L-α-tryptophan) or an unnatural amino acid (e.g., D-α-amino acid, an L-β-amino acid, a D-β-amino acid, an L-γ-amino acid, a D-γ-amino acid, an L-δ-amino acid, a D-δ-amino acid) or derivatives thereof. Each of P¹, P² and A may contain a combination of natural L-α-amino acid and unnatural amino acids, or they may each contain a single class of amino acid.

The alkenyl linker between the two modified amino acid residues of Formula I is formed from the two different alkenyl chains present on the side chains of the modified amino acid residues, as shown in Formula II. Whilst the lengths of the alkyl portions of each of the alkenyl chains are defined as n in both Formula I and Formula II, both of the n values in these formulae are independently selected from an integer between 0 and 12 (e.g., between 0 and 8, 0 and 6, 6 and 12, 4 and 10, 3 and 11, 2 and 8 or 4 and 8, or they may each independently be 0, 1, 2, 3, 4, 5, 6, 7, 8, 9, 10, 11 or 12) and need not necessarily be the same, and commonly are different. The skilled addressee would appreciate that each n value can be selected depending on the particular peptide chain sequence and the particular geometry of the folded peptide, whereby the placement of the alkene bond on the linker may influence the biology of the stapled peptide.

The stereochemistry of the peptide chain as referred to herein is defined by the relative configuration of the alkene group of the alkenyl linker, as shown by the wavy line of Formula I. Whilst other stereocentres may be found in the compounds of Formula I, such as the chiral centres of the modified amino acid residues from which the alkenyl chains are bound, the discussion herein of stereocentres and arrangements thereof refer to the configuration of atoms around the alkenyl group of the intramolecular linker. The chirality of the modified amino acids and arrangements of other stereocentres that may occur are described separately, however it should be clear that any reference to (E)- or (Z)-stereoisomers, or (E)- or (Z)-stapled peptides, refers only to the arrangement of atoms around the alkene group of the alkenyl intramolecular linker. The wavy line of Formula I is a carbon-carbon single bond that is attached to a carbon atom of the double bond. The stereochemistry of the double bond may be the (E)- (i.e., trans-) isomer or it may be the (Z)- (i.e., cis-) isomer. In other words, when the stereochemistry of a stapled peptide is described herein as a trans- or (E)-isomer, this refers to the double bond of the alkenyl chain arranged so that the alkyl groups are on opposing sides in the plane of the double bond, as shown below in Formula Ia:

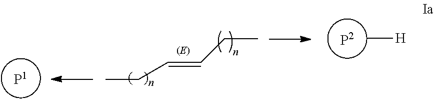

and that when the stereochemistry of a stapled peptide is described herein as a cis- or (Z)-isomer, this refers to the double bond of the alkenyl chain arranged so that the alkyl groups are on opposing sides in the plane of the double bond, as shown below in Formula Ib:

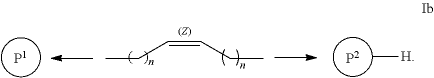

Each of the modified amino acids of the stapled peptide are also rigidized by the inclusion of an alkyl group bonded to the same carbon atom in the modified amino acid as the alkenyl side group, shown as R¹ and R² in Formula I. It is believed that the R¹ and R² groups attached to the quaternary carbon chiral centers contribute positively to the rate of reaction of forming the alkenyl linker, at least in part, due to the Thorpe-Ingold effect. The R¹ and R² groups on the quaternary carbons also effectively locks the peptide backbone in an α-helical conformation, which also promotes the alkenyl linker formation and contributes to stabilisation of the α-helix. Each R¹ and R² is an alkyl group, and may be the same or different. Each alkyl may be straight chained (e.g., methyl, ethyl, butyl, propyl or pentyl) or it may be branched (e.g., isopropyl, sec-butyl, tert-butyl, isopentyl or sec-pentyl), or it may be cycloalkyl (e.g., cyclopropyl, cyclobutyl, cyclopentyl or cyclohexyl) or heterocycloalkyl (e.g., pyrrolidinyl, thiolanyl, piperidinyl or tetrahydropyranyl). Each alkyl group may be further substituted with one or more substituents, e.g. hydroxyl, amine, halogen or thiol. As one purpose of the R¹ and R² groups is to enable self-templated formation of peptide secondary structures, there is no limitation on the substituents which would effectively provide steric hindrance to the alkenyl chain when added to the alkyl groups of R¹ and/or R². Indeed, as the Thorpe-Ingold effect increases with additional steric bulk, it may be advantageous to increase the steric bulk of the R¹ and/or R² groups.

### Catalysts/Specific Method

The process for producing a stapled peptide of Formula I, from a compound of Formula II, comprises two steps.

The first step, herein referred to as step (a), is a stereoselective metathesis reaction conducted with a compound of Formula II. This reaction links the two alkene groups to form an intramolecular alkenyl chain. By “stereoselective” it is meant that the products produced from this reaction have at least one stereocentre formed as a result of the reaction, and that a product with one particular arrangement around that stereocentre (e.g., stereoisomer) is favoured over the products (e.g., stereoisomers) with alternative arrangements around the stereocentre. It is not necessarily that only one isomer is exclusively produced, although this may occur under some conditions. However, in most cases, the stereoselective metathesis reaction disclosed herein will produce a mixture of isomers, with one isomer favoured, or produced in majority, over the others.

The compound of Formula I may be produced with a conversion of a compound of Formula II to a compound of Formula I of greater than 90% (e.g., greater than about 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9%).

The process of step (a) may result in a mixture of products, wherein the majority of that product is the less polar stereoisomer. The less polar stereoisomer may be more than about 50% of the product formed in step (a), e.g., it may be about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or 100%. The less polar stereoisomer may be the (E)-isomer (e.g., the trans-isomer). The less polar isomer is the one that elutes last in reverse-phase chromatography, as shown in FIG. 4.

The modified amino acids in the stapled peptides of Formula I that comprise the alkenyl chains to be linked each contain a chiral centre. The chiral centre is the carbon atom that has the R¹ or R² group, the amino group, the carbonyl (or carboxyl) group and the alkenyl chain bound to it, which is also referred to herein as the modified amino acid moiety. Hence, each peptide of Formula II and each stapled peptide of Formula I contain at least two chiral centres located at the modified amino acids involved in forming the intramolecular alkenyl linker. Whilst the peptide chains may have additional chiral centres in other amino acid residues or their side chains, these are not otherwise defined herein with reference to the stereochemistry of the stapled peptides. The chiral centres of the modified amino acid groups may both be (S) or they may both be (R), or one may be (S) and the other (R). The chiral centre attached to the R¹ group may be (S) or it may be (R). The chiral centre attached to the R² group may be (S) or it may be (R). The chirality of one chiral centre may or may not influence the chirality of the other chiral centre. This will depend upon the arrangement of the amino acids in the peptide chain and the length of the alkenyl chain once formed. For instance, when m (i.e., the number of amino acid residues found between the two modified amino acids) is 2, the chiral centre attached to the R¹ group may be (R) and the chiral centre of the R² group may be (S), or the chiral centre attached to the R¹ group may be (S) and the chiral centre of the R² group may be (R), or when m is 3, the chiral centre attached to the R¹ group and the R² may be both (R) or may both be (S).

The process for carrying out the reaction of step (a) is a metathesis reaction conducted in the presence of a catalyst and an organic solvent. The catalyst may contain a catalytic metal atom. It may be an anchored catalyst (e.g., a catalyst with at least one bidentate ligand coordinating to the metal atom). It may be a non-anchored catalyst (e.g., a catalyst with all monodentate ligands coordinating to the metal atom). The catalyst may be an alkylidene catalyst. It may be a non-anchored alkylidene catalyst. An alkylidene catalyst is a catalyst that catalyses reactions between alkenes. The catalyst may comprise ruthenium. It may for example be a non-anchored ruthenium catalyst (e.g., a Grubbs I catalyst or a Grubbs II catalyst), or it may be an anchored ruthenium catalyst (e.g., a Hoveyda-Grubbs I catalyst, Hoveyda-Grubbs II catalyst or a Grubbs Z catalyst). It may be a catalyst as shown in FIG. 1.

The metathesis reaction may be carried out by adding an aliquot of dissolved catalyst to a solvent containing a suspension of the peptide chain bound to a solid support, as described above as Formula II. The method of step (a) may include a single addition of an aliquot of homogenous catalyst, or it may involve multiple additions of aliquots of fresh or unused catalyst to the same reaction mixture. Where multiple aliquots are added, the number of aliquots may be 2, 3, 4, 5 or more than 5 additions of fresh or unused catalyst, before the stapled peptide is cleaved from the solid and collected. It may be three aliquots. The time between aliquot additions may be relatively short (e.g., between about 1 and 60 minutes, such as 1, 2, 3, 4, 5, 10, 15, 20, 35, 30, 35, 40, 45, 50, 55 or 60 minutes) or it may be longer (e.g., between 1 and 4 hours, such as about 1, 1.5, 2, 2.5, 3, 3.5 or 4 hours). The time between multiple aliquots may be the same throughout the method of step (a) or it may vary from aliquot to aliquot. The solvent that the catalyst is dissolved in may be the same as the solvent that the compound of Formula II is immersed in, or they may be different. The solvent used may be a halogenated alkane, for example it may be dichloroethane. If the solvents are different, they may be miscible.

The metathesis reaction of step (a) may be conducted at a temperature of between about 15° C. and about 30° C. (e.g. between about 15° C. and about 25° C., or between about 15° C. and 20° C., 20° C. and 30° C., or 20° C. and 25° C., e.g., at about 15, 16, 17, 18, 19, 20, 21, 22, 23, 24, 25, 26, 27, 28, 29 or 30° C.). The reaction may be conducted at a room temperature. The metathesis reaction of step (a) may be conducted at a temperature of between about 30° C. and about 60° C. or between about 40° C. and 60° C., about 50° C. and 60° C., about 40° C. and 50° C., about 45° C. and 55° C., about 40° C. and 55° C. about 45° C. and 60° C. and about 30° C. and 50° C., e.g., at about 40, 41, 42, 43, 44, 45, 46, 47, 48, 49, 50, 51, 52, 53, 54, 55, 56, 57, 58, 59 or 60° C.).

The metathesis reaction of the present reaction, step (a) may result in a product yield, defined by the percentage conversion of a compound of Formula II into a compound of Formula I (after cleavage), that is greater than about 55% (e.g., greater than about 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8% or 99.9%).

In the second step of the method of the present invention, herein referred to as step (b), the compound of Formula II, which is attached to a solid state resin, is cleaved from this solid state resin following the completion of step (a), resulting in the release of a compound of Formula I into solution. The cleaving of a peptide from a solid state resin support is well-known in the art. Such cleaving may be achieved by the addition of an acid, such as HF or trifluoroacetic acid, and the isolation therefrom of the liberated peptide sequence. Any method known to the skilled addressee to cleave a formed peptide from the solid support resin at the completion of solid-phase peptide synthesis may be used in this method.

### Product

A compound of Formula I may be produced by the method described herein:

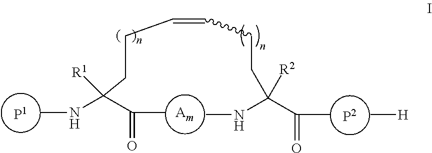

wherein:
m is an integer between 1 and 8;
each n is independently an integer between 0 and 12;
each A is independently an amino acid residue;
R¹ and R² are each independently alkyl;
[symbol] is a carbon-carbon single bond that is attached to a carbon atom of the double bond such that the compound of Formula I is in either the (E)-configuration or the (Z)-configuration or is a mixture of these; and
P¹ and P² are each independently either an amino acid residue or an oligopeptide chain or a polypeptide chain, wherein P¹ has a terminal amino group and P² has a terminal carboxyl group.

The product may comprise a compound of Formula I, wherein at least about 50% (e.g., at least about 50%, 55%, 60%, 65%, 70%, 75%, 80, 85%, 90%, 95%, 96%, 97%, 98%, 99%, 99.1%, 99.2%, 99.3%, 99.4%, 99.5%, 99.6%, 99.7%, 99.8% or 99.9%) of the product contains a carbon-carbon double bond in the (E)-stereoisomer configuration. The carbon-carbon double bond is located on the intramolecular alkylene linker.

The compound of Formula I produced by the method described herein may be a peptide analogue. The skilled addressee would understand that as the compound of Formula I contain at least two modified amino acid residues and an intramolecular alkenyl linker, it is more strictly a peptide analogue, rather than a peptide per se. The peptide analogue, or stapled peptide, may contain an α-helical region that is bridged by the intramolecular alkenyl linker, thereby stabilising the α-helical region and preventing the denaturing of this secondary structure of the stapled peptide.

The present invention may be better understood by the skilled addressee with reference to the following illustrative, and non-limiting, examples.

### EXAMPLES

### Example 1

A method of stereoselective formation of the less polar stapled peptide isomer is described below using Grubbs II catalyst in DCE. Within 2 h, >95% product conversion can be achieved. By prolonging reaction time at elevated temperatures (50° C.), E/Z selectivity may be increased (>65% d.e.).

To do this, a comprehensive solvent and catalyst screen was undertaken using linear peptide analogues ATSP-7041 (Ac-LT(tBu)F(R8)E(tBu)Y(tBu)WAQ(Trt)(cba)(S5)S(tBu)AA-NH2) and VIP116 (Ac-K(Boc)(Ahx)T(tBu)S(tBu)F(R8)E(tBu)Y(tBu)WALL(S5)E(tBu)N(Trt)F—NH2) as substrates. Accordingly, the resin-bound peptides were treated with the metathesis and solvent of choice and the reaction was allowed to progress for 2 hours before a small sample of peptide was cleaved from the resin and analysed by HPLC. This process was repeated twice using fresh catalysts. The results of each of the three cycles are displayed in Table 1 as 1 RCM (one catalyst aliquot), 2 RCM (2 catalyst aliquots) and 3 RCM (3 catalyst aliquots).

Under standard conditions with Grubbs I catalyst (entry 1, Table 2), little selectivity was observed for both stapled peptides (FIGS. 4A-B). The use of THF (entry 2, Table 2) as the solvent gave similar results whereas the use of toluene (entry 3, Table 2) had favoured the formation of the early-eluting isomer of ATSP-7041 only. Increasing the temperature greatly enhanced reactivity but no significant improvements in stereoselectivity was observed (entries 4-5, Table 2).

TABLE 2
Ruthenium-or molybdenum-catalysed ring-closing metathesis to form i, i + 7 stapled peptides.

| | | ATSP-7041 | | | | | | VIP116 | | | | | |
|---|---|---|---|---|---|---|---|---|---|---|---|---|---|
| | | 1 RCM | | 2 RCM | | 3 RCM | | 1 RCM | | 2 RCM | | 3 RCM | |
| | | conv^{b} | d.e.^{c} | conv | d.e. | conv | d.e. | conv | d.e. | conv | d.e. | conv | d.e. |
| Entry | Metathesis conditions^{a} | (%) | (%) | (%) | (%) | (%) | (%) | (%) | (%) | (%) | (%) | (%) | (%) |
| 1 | G1, DCE, rt | 47 | −5 | >95 | −26 | >95 | −20 | 75 | −20 | >95 | −5 | >95 | −9 |
| 2 | G1, THF, rt | 44 | −26 | 68 | −29 | 74 | −35 | 46 | −13 | 63 | −13 | 81 | −17 |
| 3 | G1, toluene, rt | 23 | −44 | 74 | −41 | 75 | −39 | 38 | 5 | 69 | −5 | 78 | −5 |
| 4 | G1, DCE, 50° C. | >95 | −17 | >95 | −9 | >95 | −9 | >95 | −5 | >95 | 0 | >95 | 5 |
| 5 | G1, toluene, 50° C. | 42 | −31 | 86 | −29 | 94 | −29 | 59 | −13 | 84 | −17 | 88 | −5 |
| 6 | G2, DCE, rt | >95 | 57 | >95 | 57 | >95 | 57 | >95 | 39 | >95 | 57 | >95 | 61 |
| 7 | G2, DCE, 50° C. | >95 | 52 | >95 | 60 | >95 | 70 | >95 | 44 | >95 | 56 | >95 | 66 |
| 8 | HG1, DCE, rt | 14 | −31 | 57 | −26 | 71 | −13 | 23 | 9 | 59 | 0 | 66 | 5 |
| 9 | HG1, DCE, 50° C. | 61 | −20 | 87 | −20 | 94 | −17 | 63 | −5 | 86 | −9 | 93 | −5 |
| 10 | HG2, DCE, rt | 49 | 47 | 81 | 47 | 84 | 39 | 49 | 29 | 63 | 17 | 69 | 20 |
| 11 | HG2, DCE, 50° C. | 91 | 56 | >95 | 66 | >95 | 59 | 87 | 29 | 94 | 31 | 97 | 52 |
| 12 | CatPac-1, toluene, 60° C. | — | — | — | — | — | — | — | — | — | — | — | — |
| 13 | CatPac-3, toluene, 60° C. | — | — | — | — | — | — | — | — | — | — | — | — |
| 14 | CatPac-1, toluene, 60° C.^{d} | — | — | — | — | — | — | — | — | — | — | — | — |
| 15 | CatPac-3, toluene, 60° C.^{d} | — | — | — | — | — | — | — | — | — | — | — | — |

^{a}G1 = Grubbs I, G2 = Grubbs II, HG1 = Hoveyda-Grubbs I, HG2 = Hoveyda-Grubbs II.
^{b}Percent conversion = product/(product + starting material) as determined by reverse phase HPLC.
^{c}Selectivity for the less polar isomer = (late eluting isomer − early eluting isomer)/(late eluting isomer + early eluting isomer).
^{d}The reaction was carried out in solution phase.

Grubbs II catalyst on the other hand enabled complete ring-closing metathesis at room temperature within 2 hours (entries 6-7, Table 2). Prolonging the reaction does not reverse the inherent selectivity but may increase product formation of the desired, late-eluting isomer (FIGS. 4C-F).

Interestingly, ring-closing metathesis of linear ATSP-7041 as per the conditions reported by Aileron Therapeutics revealed Hoveyda-Grubbs II catalyst also displayed good selectivity (entry 11, Table 1). However, ruthenium catalysts with anchored alkylidene groups (entries 8-11, Table 2) in general are less reactive and selective than Grubbs II catalyst.

To assess the reactivity of the CatPac catalysts, a trial reaction was first conducted using allylbenzene (Scheme 1). Following careful purging of the Schlenk tube with Argon, the reaction vessel containing allylbenzene and CatPac-1 was charged with toluene and heated to 60° C., with periodic evacuation of the vessel atmosphere to remove any formed ethylene gas. After stirring for 16 hours, the desired product was successfully attained.

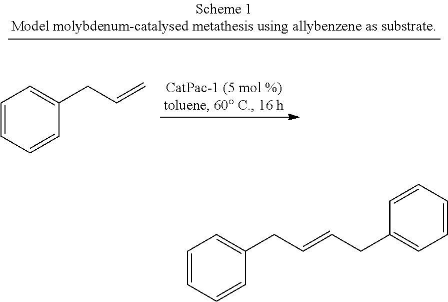

The reaction conditions were repeated using resin-bound, linear ATSP-7041 and VIP116 with either CatPac-1 or CatPac-3. However, no appreciable product formation was observed (entries 12-13, Table 2). To ascertain that the failure of the reaction wasn't due to inefficient reagent mixing (the reaction was carried out under diffusion conditions to prevent damage to the resin), cleaved and lyophilised peptides were next used as the substrate. Unfortunately, rapid stirring under solution phase conditions also failed (entries 14-15, Table 2).

Although molybdenum offers the advantage of non-toxicity to the human body, we have found the paraffin coated Mo catalysts to be incompatible with our peptide systems. Furthermore, stringent purging and Schlenk techniques may be arduous to the non-chemists in the stapled peptide community. In contrast, we have demonstrated acceptable levels of selectivity during the macrocyclisation step using Grubbs II catalyst, the setup of which can be conveniently adapted in all laboratories.

The current invention has been trialed on 5 p53-targeting stapled peptides using in-house catalysts. The technology is tolerant of a range of amino acids.

### Example 2

In order to assess whether the geometry of the hydrocarbon bridge had an effect on the biological activities of stapled peptides, the cis and trans isomers of several MDM2-targeting peptides were synthesised via conventional manner (Grubbs I, DCE) followed by careful reverse-phase HPLC separation.

The binding affinity of each stapled peptide isomer to MDM2 was determined using a fluorescence anisotropy assay. In all cases, the binding of the late-eluting, less polar stapled peptide isomer, the trans stereoisomer, was equal to or stronger than that of the early-eluting isomer (Table 3).

TABLE 3
Binding affinities of p53-derived stapled peptides to MDM2.

| Name | Peptide Sequence | | | | | | | | | | | | | | | | | | | | (Kd, nM) |
|---|---|---|---|---|---|---|---|---|---|---|---|---|---|---|---|---|---|---|---|---|---|
| PM2-Early | Ac | | | | | T | S | F | R8 | E | Y | W | A | L | L | S5 | NH2 | | | | 57.1 ± 9.8 |
| PM2-Late | Ac | | | | | T | S | F | R8 | E | Y | W | A | L | L | S5 | NH2 | | | | 26.4 ± 5 |
| VIP116-Early | Ac | | | K | Ahx | T | S | F | R8 | E | Y | W | A | L | L | S5 | E | N | F | NH2 | 11.3 ± 2.2 |
| VIP116-Late | Ac | | | K | Ahx | T | S | F | R8 | E | Y | W | A | L | L | S5 | E | N | F | NH2 | 9.9 ± 1.9 |
| VIP82-Early | Ac | | K | K | Ahx | T | S | F | R8 | E | Y | W | A | L | L | S5 | E | N | F | NH2 | 20.4 ± 1.6 |
| VIP82-Late | Ac | | K | K | Ahx | T | S | F | R8 | E | Y | W | A | L | L | S5 | E | N | F | NH2 | 13.4 ± 0.9 |
| VIP115-Early | Ac | K | K | K | Ahx | T | S | F | R8 | E | Y | W | A | L | L | S5 | E | N | F | NH2 | 16.1 ± 1.3 |
| VIP115-Late | Ac | K | K | K | Ahx | T | S | F | R8 | E | Y | W | A | L | L | S5 | E | N | F | NH2 | 13.8 ± 1 |

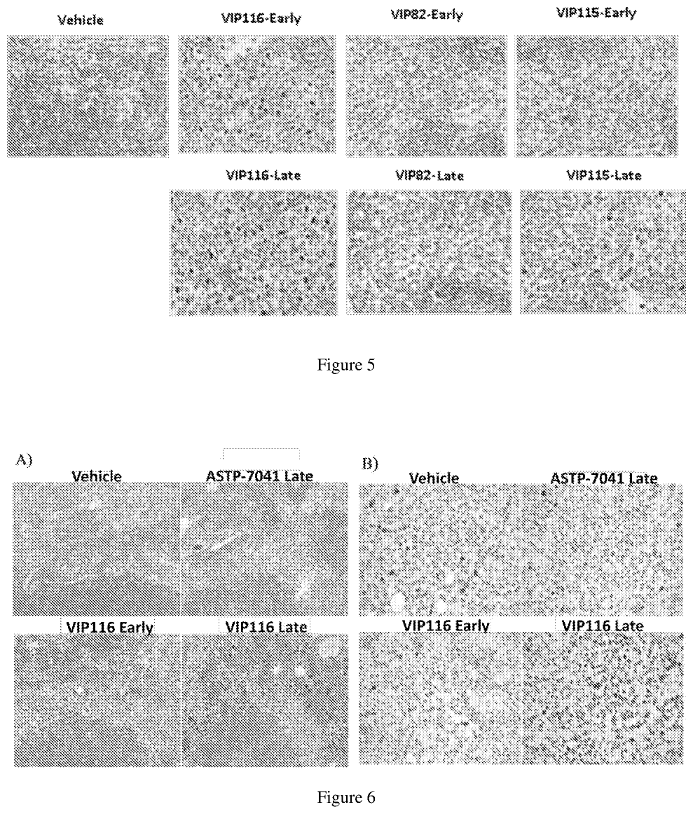

A subset of the peptide analogues was subsequently assessed for their ability to stabilise p53 protein levels in proliferating cells. In particular, lysine-containing peptides were chosen due to their enhanced solubility in biological buffers. Normal p53 wild type mice were administered with either vehicle (DMSO) or 40 mg/kg of peptide via IP. Spleens were then harvested 5 hours post injection and histological samples were processed for p53 staining (FIG. 5).

As expected, mice treated with DMSO vehicle did not show appreciable p53 protein levels. For VIP82 and VIP115, the late-eluting isomer attained a higher concentration of p53 in mouse tissues compared to the early-eluting isomer. For VIP116, the late-eluting isomer was as active as the early-isomer.

In all cases, stereoselective synthesis of the less-polar stereoisomer using the described invention would lead to a peptide that is at least equal to, and usually more potent than, the cisltrans mixture.

### Example 3

The stapled peptides VIP116 (both early-eluting and late-eluting isomers) and ATSP-7041 (late-eluting isomer) were injected into C57BL/6 mice bearing an allograft tumour (B16F10 melanoma cells) via intraperitoneal injection at a dose of 40 mg/kg. The spleen and tumour were both harvested from the mice 6 hours post injection. The p53 stabilization was detected and visualised in harvested tissue samples by p53 IHC staining (FIG. 6). A vehicle (2% DMSO in HBSS buffer) was used as a control.

As can be seen from FIG. 6, compared to the early isomer, the late-eluting isomer of VIP116 exhibited a higher p53 activation response in both spleen and tumour samples taken from the mice. Furthermore, the activity of the late-eluting isomer of VIP116 was more potent than the late-eluting isomer of ATSP-7041, a current pre-clinical candidate.

1. A process for producing a compound of Formula I 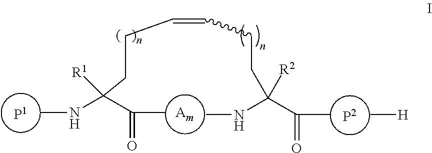
said process comprising the steps of:
a) performing a stereoselective metathesis reaction on a compound of Formula II 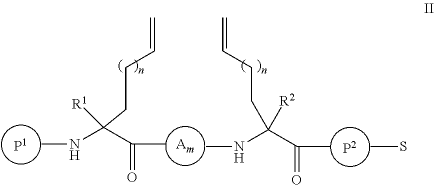
so as to form an intramolecular alkenyl chain, and;
b) cleaving S from P² so as to produce a compound of Formula I;
wherein:
m is an integer between 1 and 8;
each n is independently an integer between 0 and 12;
each A is independently an amino acid residue;
R¹ and R² are each independently alkyl;
P¹ and P² are each independently an amino acid residue or an oligopeptide chain or a polypeptide chain, wherein P¹ has a terminal amino group and P² has a terminal carboxyl group; [symbol]
is a carbon-carbon single bond that is attached to a carbon atom of the double bond such that the compound of Formula I is in either the (E)-isomer configuration or the (Z)-isomer configuration or is a mixture of these; and,
S is a solid state resin.

2. The process of claim 1, wherein the compound of Formula (I) is produced with a geometric stereoisomer purity of greater than 50%.

3. The process of claim 1 wherein more than about 50% of the product formed in step a) is the less-polar stereoisomer.

4. The process of claim 3, wherein the less-polar stereoisomer is the (E)-stereoisomer.

5. The process of any one of claims 1 to 4 wherein m is an integer between 1 and 6.

6. The process of any one of claims 1 to 5 wherein m is 6.

7. The process of any one of claims 1 to 5, wherein when m is 2, the chiral centre attached to R¹ is (R) and the chiral centre attached to R² is (S).

8. The process of any one of claims 1 to 5, wherein when m is 3, the chiral center attached to R¹ and the chiral center attached to R² are either both (R) or both (S).

9. The process of any one of claims 1 to 8 wherein each A independently is a naturally occurring L-α-amino acid.

10. The process of any one of claims 1 to 8 wherein A comprises at least one unnatural amino acid or a derivative thereof.

11. The process of any one of claims 1 to 10 wherein R¹ is methyl.

12. The process of any one of claims 1 to 11 wherein R² is methyl.

13. The process of any one of claims 1 to 12 wherein S comprises a polymeric material.

14. The process of any one of claims 1 to 13 wherein S is a polyethylene glycol resin.

15. The process of any one of claims 1 to 14 wherein P¹ comprises at least one naturally occurring L-α-amino acid.

16. The process of any one of claims 1 to 15 wherein P¹ comprises at least one unnatural amino acid or a derivative thereof.

17. The process of any one of claims 1 to 16 wherein P² comprises at least one naturally occurring L-α-amino acid.

18. The process of any one of claims 1 to 17 wherein P² comprises at least one unnatural amino acid or a derivative thereof.

19. The process of any one of claims 1 to 18 wherein step a) is conducted in the presence of a catalyst and an organic solvent.

20. The process of claim 19 wherein the catalyst is a non-anchored catalyst.

21. The process of claim 20, wherein the catalyst is a non-anchored alkylidene catalyst.

22. The process of any one of claims 19 to 21 wherein the catalyst comprises ruthenium.

23. The process of claim 22 wherein the catalyst is Grubbs II.

24. The process of any one of claims 19 to 23 wherein fresh catalyst is added to the compound of Formula II in separate aliquots.

25. The process of claim 24 wherein fresh catalyst is added to the compound of Formula II 2, 3, 4 or 5 times, before conducting step b).

26. The process of any one of claims 19 to 25 wherein the organic solvent is a halogenated alkane.

27. The process of claim 26 wherein the organic solvent is dichloroethane.

28. The process of any one of claims 1 to 27 wherein step a) is conducted at a temperature between about 15° C. and about 30° C.

29. The process of any one of claims 1 to 27 wherein step a) is conducted at a temperature between about 40° C. and about 60° C.

30. The process of any one of claims 1 to 29 wherein step a) converts at least about 55% of the compound of Formula II into the compound of Formula I.

31. The process of any one of claims 1 to 30 comprising the step of producing the compound of Formula II by solid-phase peptide synthesis prior to step a).

32. The process of claim 31 wherein the combined number of amino acid residues in P¹ and P² plus m plus 2 is between 5 and 20.

33. A product obtained by the process of any one of claims 1 to 32 which comprises a compound of Formula I as defined in claim 1.

34. The product of claim 33 wherein at least about 50% of the product contains a carbon-carbon double bond in the (E)-stereoisomer configuration.

35. The product of claim 33 or claim 34 wherein the compound of Formula I is a peptide analogue stabilised in an α-helical conformation.
